(19) United States
(12) Patent Application Publication
(10) Pub. No.: US 2025/0127354 A1
(43) Pub. Date: Apr. 24, 2025

(54) COMBINATION OF A FLOOR CLEANER AND A CLEANING APPARATUS FOR CLEANING THE FLOOR CLEANER

(71) Applicant: Techtronic Cordless GP, Anderson, SC (US)

(72) Inventors: Patrick Quinn, Huntersville, NC (US); Roger Donovan Bode, Charlotte, NC (US)

(21) Appl. No.: 18/918,290

(22) Filed: Oct. 17, 2024

Related U.S. Application Data
(60) Provisional application No. 63/591,803, filed on Oct. 20, 2023.

(51) Int. Cl. A47L 9/04; A47L 9/28; A47L 9/30

(52) U.S. Cl. CPC A47L 9/0477; A47L 9/0411; A47L 9/2847; A47L 9/30

(57) ABSTRACT

A combination of a floor cleaner and a cleaning apparatus for cleaning the floor cleaner is disclosed. The floor cleaner includes a base movable along a surface to be cleaned, a brushroll motor, and a brushroll rotatable about a brushroll axis relative to the base by the brushroll motor. The brushroll has a brushroll radius defined from the brushroll axis to an outer perimeter of the brushroll. The cleaning apparatus includes a tray that selectively removably receives a portion of the floor cleaner in a mounted position. The tray includes a brushroll-receiving portion to receive the brushroll of the floor cleaner. The combination further includes a light source directed toward the brushroll when the brushroll is received in the brushroll-receiving portion of the tray. The light source is operable to emit ultraviolet light onto the brushroll, the ultraviolet light having a wavelength less than 400 nm.

### CROSS-REFERENCE TO RELATED APPLICATIONS

This application claims priority to U.S. Provisional Patent Application No. 63/591,803, filed Oct. 20, 2023, the entire contents of which are hereby incorporated by reference herein.

### BACKGROUND

The present disclosure relates to a floor cleaner, and more particularly to a floor cleaner having a corresponding cleaning apparatus for cleaning the floor cleaner, including a cleaning apparatus that uses light and/or air to clean the floor cleaner.

### SUMMARY

In one embodiment, a combination of a floor cleaner and a cleaning apparatus for cleaning the floor cleaner is disclosed. The floor cleaner includes a base movable along a surface to be cleaned, a brushroll motor, and a brushroll having a length along a brushroll axis, the brushroll rotatable about the brushroll axis relative to the base by the brushroll motor. The brushroll has a brushroll radius defined from the brushroll axis to an outer perimeter of the brushroll. The cleaning apparatus includes a tray configured to selectively removably receive a portion of the floor cleaner in a mounted position. The tray includes a brushroll-receiving portion to receive the brushroll of the floor cleaner. The combination further includes a light source directed toward the brushroll when the brushroll is received in the brushroll-receiving portion of the tray. The light source is operable to emit ultraviolet light onto the brushroll, the ultraviolet light having a wavelength less than 400 nm.

In another embodiment, a cleaning apparatus for a floor cleaner is disclosed. The floor cleaner includes a base movable along a surface to be cleaned, a brushroll motor, a brushroll rotatable about a brushroll axis relative to the base by the brushroll motor, and a component that is removable from the floor cleaner. The cleaning apparatus includes a housing defining a compartment configured to receive the component at a component-receiving location when the component is removed from the floor cleaner. The cleaning apparatus further includes a cleaning member within the compartment. The cleaning member is directed toward the component-receiving location and is selectively operable in at least one of a cleaning mode or a drying mode.

In another embodiment, a method of cleaning a floor cleaner by using a cleaning apparatus is disclosed. The method includes operating a light source to direct ultraviolet light toward an exposed interior of a brushroll of the floor cleaner, and the method further includes rotating the brushroll at a desired rotational speed while the ultraviolet light is directed toward the exposed interior of the brushroll to achieve a desired dwell time of the ultraviolet light in the brushroll.

Other aspects of the disclosure will become apparent by consideration of the detailed description and accompanying drawings.

### BRIEF DESCRIPTION OF THE DRAWINGS

FIG. 1 is a perspective view of a floor cleaner in a mounted position in a cleaning apparatus according to an embodiment.

FIG. 2 is an exploded view of the floor cleaner and the cleaning apparatus of FIG. 1.

FIG. 3 is an upper perspective view of the cleaning apparatus of FIG. 1.

FIG. 4 is a cross-section of the floor cleaner in the mounted position in the cleaning apparatus of FIG. 1 along the line A-A.

FIG. 5 is a cross-section of a floor cleaner in a mounted position in a cleaning apparatus according to another embodiment.

FIG. 6 is a cross-section of a floor cleaner in a mounted position in a cleaning apparatus according to another embodiment.

FIG. 7 is a cross-section of a floor cleaner in a mounted position in a cleaning apparatus according to another embodiment.

FIG. 8 is a cross-section of a floor cleaner in a mounted position in a cleaning apparatus according to another embodiment.

FIG. 9 is a plan view of a wiper for use with the cleaning apparatus of FIG. 1 according to an embodiment.

FIG. 10 is a cross-section of a floor cleaner in a mounted position in a cleaning apparatus having a fan to deliver air to a brushroll of the floor cleaner according to an embodiment.

FIG. 11 is a cross-section of a floor cleaner in a mounted position in a cleaning apparatus having a fan to deliver air to a brushroll of the floor cleaner according to another embodiment.

FIG. 12 is an upper perspective view of a cleaning apparatus showing an air opening according to an embodiment.

FIG. 13 is an upper perspective view of a cleaning apparatus showing a plurality of air openings according to another embodiment.

FIG. 14 is an upper perspective view of a cleaning apparatus showing a plurality of air openings according to another embodiment.

FIG. 15 is a perspective view of a cleaning apparatus for use with the floor cleaner of FIG. 1 according to another embodiment, with a door in an open position.

FIG. 16 is a cross-section of the cleaning apparatus of FIG. 15 along the line B-B, with the door in a closed position.

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

In addition, it should be understood that embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects may be implemented in software (e.g., stored on non-transitory computer-readable medium) executable by one or more processing units, such as a microprocessor and/or application specific integrated circuits (“ASICs”). As such, it should be noted that a plurality of hardware and software-based devices, as well as a plurality of different structural components, may be utilized to implement the embodiments. For example, “servers,” “computing devices,” “controllers,” “processors,” etc., described in the specification can include one or more processing units, one or more computer-readable medium modules, one or more input/output interfaces, and various connections (e.g., a system bus) connecting the components.

### DETAILED DESCRIPTION

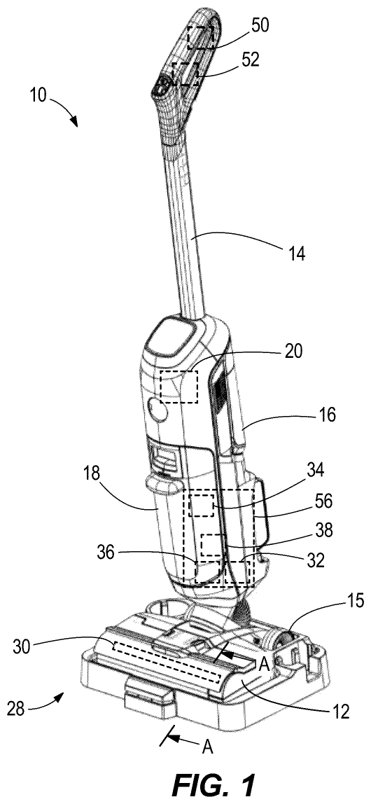

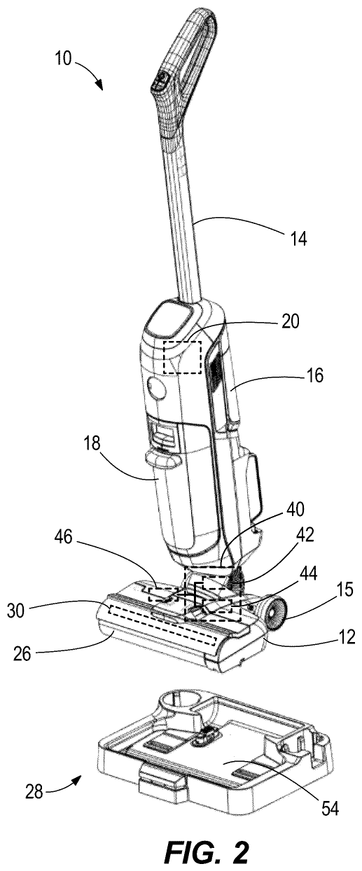

FIG. 1 illustrates a floor cleaner 10 positioned in a cleaning apparatus 28 that is formed as a cleaning tray in the illustrated embodiment. After a surface or floor cleaning operation of the floor cleaner 10, the floor cleaner 10 can be placed in the cleaning apparatus 28 to clean a brushroll 26 (FIG. 2) of the floor cleaner 10 and/or other portions of the floor cleaner 10. The cleaning apparatus 28 includes a light source configured to direct ultraviolet light onto the floor cleaner 10 to contribute to cleaning or sanitizing of the floor cleaner 10.

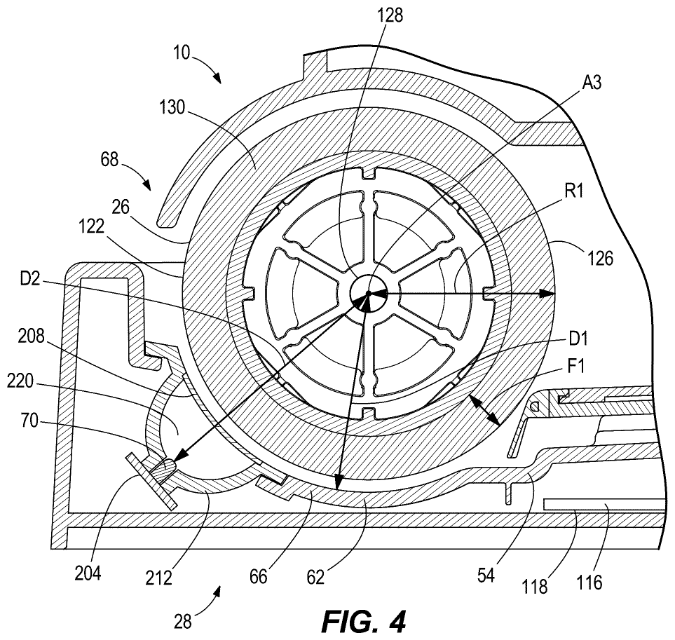

In the illustrated embodiment, the floor cleaner 10 includes a base 12 and an upright portion 14 pivotally coupled to the base 12. The illustrated floor cleaner 10 includes wheels 15 rotatably attached to the base 12 for moving the base 12 over a floor surface. With reference to FIGS. 2 and 4, the floor cleaner 10 includes at least one brushroll 26 disposed in a brushroll housing 236 in the base 12 and rotatable with respect to the base 12. A brushroll motor 46, illustrated schematically in FIG. 1, is disposed in the floor cleaner 10 operably connected to the brushroll 26 and configured to rotate the brushroll 26, such as by a belt, a gear drive, or another brushroll drive. The upright portion 14 is movable and, more specifically, pivotal relative to the base 12 between an upright storage position (FIG. 1) in which the upright portion 14 is maintained in an upright position by the base 12 and an inclined use position in which the upright portion 14 is tilted rearwardly with respect to the upright storage position. In the illustrated embodiment, the floor cleaner 10 includes a supply tank 16, a recovery tank 18, and a suction motor 20. The supply tank 16 stores a cleaning fluid such as water or a cleaning solution. The floor cleaner 10 dispenses the cleaning fluid onto a surface to be cleaned through a fluid distributor 56. The fluid distributor 56, shown schematically in FIG. 1, may include a pump 34 and/or a valve 38 operably connected to the supply tank 16 and that controls the dispensation of the cleaning fluid from the supply tank 16 during operation. The fluid distributor 56 further includes a distribution nozzle 32 and/or an auxiliary fluid outlet 36 through which the cleaning fluid may be dispensed. The base 12 further includes a suction inlet 30 in fluid communication with the recovery tank 18 and the suction motor 20 operable to draw air and dispensed cleaning fluid into the suction inlet 30. The cleaning fluid is drawn from the surface to be cleaned through the suction inlet 30 and into the recovery tank 18 during operation of the floor cleaner 10. In one embodiment, the suction motor 20 also drives the brushroll 26 such that the brushroll motor 46 is the suction motor 20.

With continued reference to FIG. 1 and with reference to FIG. 2, the cleaning apparatus 28 is configured for the base 12 to be placed in the cleaning apparatus 28 for cleaning and/or storage of the floor cleaner 10. The cleaning apparatus 28 forms a tray 54 configured to receive cleaning fluid as well as to receive at least a portion of the base 12 of the floor cleaner 10 when the base 12 is placed in the cleaning apparatus 28. The cleaning apparatus 28 receives the floor cleaner 10 in a mounted position when the floor cleaner 10 or a portion of the floor cleaner 10 is operably connected to the cleaning apparatus 28 (for example, when the base 12 or a portion of the base 12 is placed on or in the tray 54) for cleaning or storage or another operation.

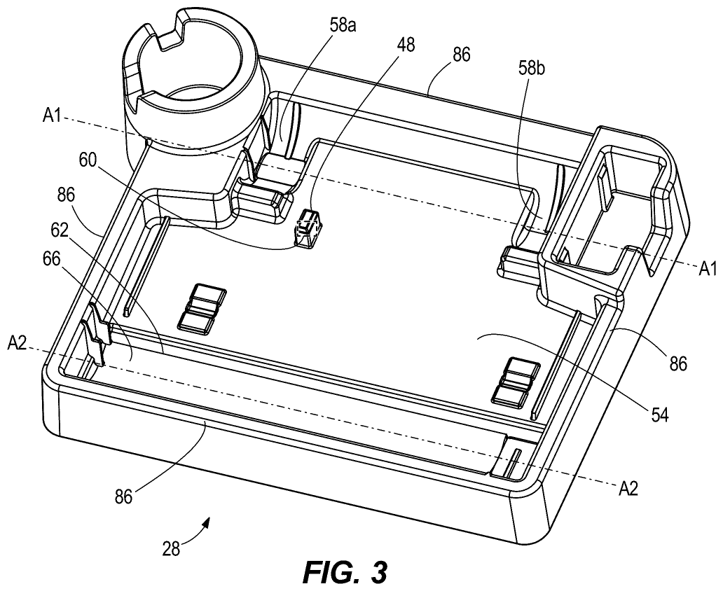

With reference to FIG. 3, the cleaning apparatus 28 may further include wheel-receiving portions 58a, 58b that are spaced from one another and that receive the wheels 15 of the floor cleaner 10 when the floor cleaner 10 is in the cleaning apparatus 28. The wheel-receiving portions 58a, 58b may define a wheel-receiving axis Al passing through a center, and in some embodiments a center of curvature, of the wheel-receiving portions 58a, 58b. The cleaning apparatus 28 includes a brushroll-receiving portion 62 that receives the brushroll 26 when the floor cleaner 10 is in the cleaning apparatus 28. The brushroll-receiving portion 62 includes a lower portion 66 that accommodates the brushroll 26 when the brushroll 26 is positioned in the tray 54. In the illustrated embodiment, the lower portion 66 includes a concave portion that matches the brushroll shape, such as a convex portion 122 of the brushroll 26 (described herein with reference to FIG. 4); however, the lower portion 66 may be a flat surface or any other shaped surface as desired. The brushroll-receiving portion 62 defines a longitudinal axis A2 of the brushroll-receiving portion 62 passing through a location of the brushroll 26 when the brushroll 26 is positioned in the tray 54. The brushroll-receiving portion 62 is positioned corresponding to the position of the brushroll 26 when the floor cleaner 10 is in the cleaning apparatus 28, which in the illustrated embodiment is on an opposite side of the cleaning apparatus 28 from the wheel-receiving portions 58a, 58b. In the illustrated embodiment, the longitudinal axis A2 of the brushroll-receiving portion 62 is parallel to or generally parallel to the wheel-receiving axis A1. The cleaning apparatus 28 may include a wall system 86 at a border of the tray 54 to, among other things, collect liquid and inhibit liquid from leaving the tray 54 except in a controlled manner (for example, through a suction system of the floor cleaner 10 or through a drain, or by a user pouring the liquid out).

With reference to FIG. 3, in some embodiments, the cleaning apparatus 28 may include an ultraviolet light generator 68 including a light source 70 (FIGS. 4-8) to generate ultraviolet light and to direct the ultraviolet light toward the brushroll 26. The ultraviolet light that is delivered to the brushroll 26 may partially or wholly sanitize the brushroll 26. Ultraviolet light may include light having a wavelength selected between 100 nm and 400 nm in some embodiments, and in other embodiments may include light having a wavelength selected between 200 nm and 400 nm. In some embodiments, the UV light has a wavelength selected between 200 nm and 300 nm, and may be selected for a desired germicidal effectiveness, such as a UV-C wavelength.

With reference to FIG. 4, the cleaning apparatus 28 includes the light source 70 extending longitudinally along at least a portion of the length of the brushroll. In the illustrated embodiment, the light source 70 includes a plurality of light emitting diodes (“LEDs”) mounted to a light bar 204 that extends along a length of the brushroll 26. In other embodiments, the light source 70 includes a UV bulb, a UV tube, a UV lamp, or another type of UV generating light source that emits ultraviolet light at the selected wavelength. In the embodiment of FIG. 4, the cleaning apparatus 28 further includes a lens 208, cleaning apparatus control circuitry 116, and a reflector 212. The reflector 212 may be made of aluminum or another material that reflects ultraviolet light, or may be molded plastic having a UV reflective coating. In some embodiments, the light source 70 is installed in the cleaning apparatus 28 omitting the reflector 212 when reflection is not desired or is unneeded to direct the UV light; for example, in some embodiments, the reflector 212 may be omitted when the light source 70 includes an LED configured to provide a beam having a direction and beam angle configured as desired for the application. Alternatively or additionally, the lens 208 may be omitted when not desired or when unneeded to cover the light source 70. The lens 208 may be used to cover the light source 70 from water, for example.

With continued reference to FIG. 4, the cleaning apparatus 28, and more specifically the cleaning apparatus control circuitry 116, includes a printed circuit board assembly (“PCBA”) 118. The PCBA 118 may be positioned on a bottom side of the cleaning apparatus 28 or in another position, and the PCBA 118 may be positioned in a location configured to inhibit water ingress. In the illustrated embodiment, the PCBA 118 is positioned inside the tray 54. The cleaning apparatus control circuitry 116 is configured to control operation of the cleaning apparatus 28 including, among other things, the light source 70. In some embodiments, the PCBA 118 includes a controller. In some embodiments, the PCBA 118 is configured to operably connect to control circuitry in the floor cleaner 10 to receive one or more signals from the floor cleaner 10 representative of characteristics of, attributes of, and/or inputs from the floor cleaner 10 and send one or more signals to the floor cleaner 10 representative of characteristics of, attributes of, and/or inputs from the cleaning apparatus 28. The PCBA 118 is configured to control the cleaning apparatus 28 and/or the floor cleaner 10 based at least in part on the one or more signals received from the floor cleaner 10.

In some embodiments, the cleaning apparatus control circuitry 116 includes a controller. The controller is electrically and/or communicatively connected to a variety of modules or components of the cleaning apparatus 28. The controller includes a plurality of electrical and electronic components that provide power, operational control, and protection to the components and modules within the controller and/or the cleaning apparatus 28. The controller further includes an electronic processor (for example, a microprocessor or another suitable programmable device) and memory. The memory includes, for example, a program storage area and a data storage area. The program storage area and the data storage area can include combinations of different types of memory, such as read-only memory (ROM) and/or random access memory (RAM). Various non-transitory computer readable media, for example, magnetic, optical, physical, or electronic memory may be used. The electronic processor is communicatively coupled to the memory and executes software instructions that are stored in the memory, or stored on another non-transitory computer readable medium such as another memory or a disc. The software may include one or more applications, program data, filters, rules, one or more program modules, and other executable instructions. The controller receives a signal(s) indicative of a characteristic(s) and controls the cleaning apparatus 28 accordingly (for example, by varying a control signal). Although the controller is described as one controller, the controller could also include multiple controllers configured to work together to achieve a desired level of control for the cleaning apparatus 28. As such, any control functions and processes described herein with respect to the controller could also be performed by two or more controllers functioning in a distributed manner.

With continued reference to FIG. 4, the brushroll-receiving portion 62 receives the brushroll 26 when the floor cleaner 10 is in the cleaning apparatus 28. The brushroll 26 has a length defining a longitudinal axis A3 of rotation, shown perpendicular to or generally perpendicular to the frame of reference of FIG. 4. The brushroll 26 has an outer perimeter 126, and in some embodiments, the brushroll 26 is shaped as a cylinder. The brushroll 26 has a radius R1 defined from the longitudinal axis A3 of the brushroll 26 to the outer perimeter 126 of the brushroll. In some embodiments, the longitudinal axis A3 of the brushroll 26 is parallel to and/or collinear with the longitudinal axis A2 of the brushroll-receiving portion 62. The brushroll 26 includes an operational material 130 (e.g., bristles, tufted fiber pile, microfiber, woven or non-woven fabric, sponge, etc.) connected to or extending from a central brushroll dowel 128, the operational material 130 forming the outer perimeter 126 of the brushroll 26.

With continued reference to FIG. 4, a first distance D1 is radially defined between the longitudinal axis A3 of the brushroll 26 and the lower portion 66 when the brushroll 26 is received by the brushroll-receiving portion 62. A second distance D2 is radially defined between the longitudinal axis A3 of the brushroll 26 and/or between the longitudinal axis A2 of the brushroll-receiving portion 62 and the light source 70. For example, in the illustrated embodiment, the second distance D2 is radially defined between the longitudinal axis A3 of the brushroll. 26 and the light source 70. In the illustrated embodiment, D2 is greater than D1. In some embodiments, D2 is equal to D1. In other embodiments, D2 is less than D1. The second distance D2 is selected such that the light source 70 is a desired distance from the brushroll 26 for the ultraviolet light emitted from the light source 70 to provide the desired cleaning or sanitizing effect for the application, for one example, inhibiting microbial growth on the outer perimeter 126 of the brushroll 26.

With continued reference to FIG. 4, the lens 208 is a UV transmitting material such as quartz glass, fused silica glass, or other UV transmitting material, i.e., a material allowing ultraviolet light at the selected wavelength to pass through, and is positioned between the light source 70 and the brushroll 26 to cover the light source 70. For some wavelengths of UV light, UV transmitting acrylic, polycarbonate, or other polymers may be selected. In some embodiments, the lens 208 may seal with the tray 54, the reflector 212, or other components to inhibit moisture from a lighting cavity 220 that is defined in the space between the lens 208 and the light source 70. The lens 208 may have a curvature that matches a curvature of, for example, the lower portion 66, or may be flat, or convex, or may have another shape as desired. The reflector 212 forms a concave shape, an angled shape, or another desired shape adjacent the light source 70 and is configured to reflect a desired amount of UV light. The reflector 212 is configured by its shape and surface properties to reflect ultraviolet light emitted from the light source 70 toward the brushroll 26.

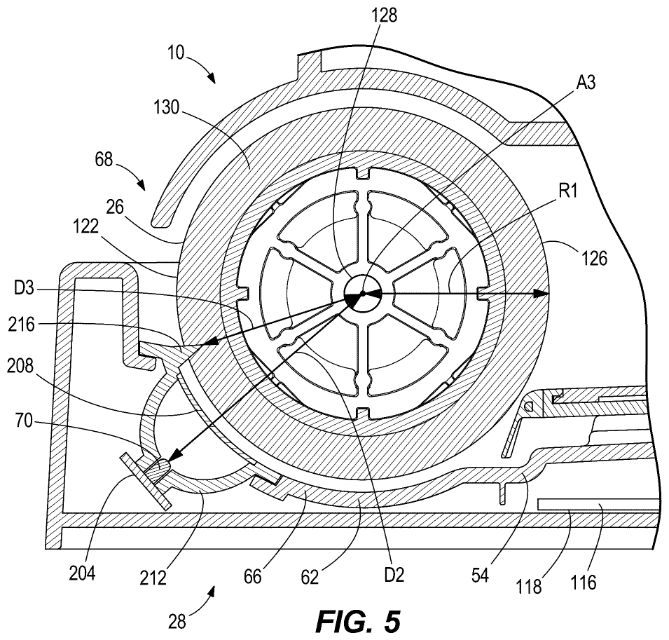

With reference to FIG. 5, an embodiment of the cleaning apparatus 28 includes a wiper 216 that is formed as a projection for engaging the brushroll 26 adjacent the light source. A third distance D3 is radially defined between the longitudinal axis A3 of the brushroll 26 and/or between the longitudinal axis A2 of the brushroll-receiving portion 62 and the wiper 216. The third distance D3 is less than the radius R1 such that the wiper 216 interferes with the brushroll 26 to deflect the operational material 130, for example to deflect or separate fibers or bristles of the brushroll 26, and thereby to allow ultraviolet light to be directed into the brushroll 26, e.g. onto the operational material 130 inside the outer perimeter 126, when the brushroll 26 is received within the brushroll-receiving portion 62 (e.g., when the base 12 is in the cleaning apparatus 28). In some embodiments, the third distance D3 may be selected, for example, between 0.5 mm and 12 mm less than the radius R1, between 1 mm and 8 mm less than the radius R1, or between 2 mm and 6 mm less than the radius R1. In some embodiments, the operational material 130 includes fibers having a fiber length F1 (shown in FIG. 4, for example), e.g., the distance the fibers extend from the brushroll dowel to the outer perimeter 126, and the difference between the radius R1 and the third distance D3 is a function of the fiber length F1, for example, R1 minus D3 equals between 10% and 95% of the fiber length F1, or between 20% and 60% of the fiber length F1, or between 50% and 90% of the fiber length F1.

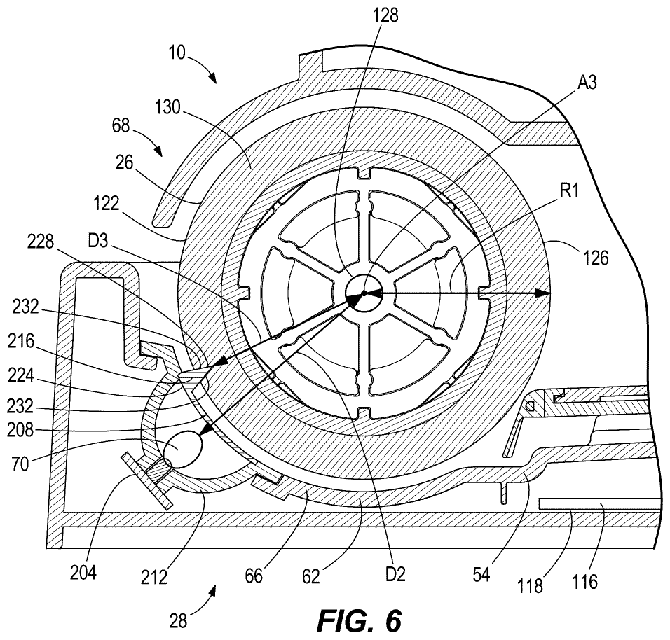

In the illustrated embodiment, the wiper 216 is provided adjacent to the light source 70. The wiper 216 is configured to cooperate with the rotation of the brushroll 26 so that the wiper 216 exposes an interior of the brushroll 26, within the operational material 130 inside the outer perimeter 126, to the ultraviolet light that is emitted from the light source 70 as the brushroll 26 rotates. For example, the brushroll 26 may rotate in a counterclockwise direction in the orientation of FIG. 5. In the illustrated embodiment, the wiper 216 is an extension of the tray 54. The ultraviolet light that is emitted from the light source 70 passes through the lens 208 and into a portion of the operational material 130 of the brushroll 26 (i.e., into a portion of the brushroll 26 that is exposed by the wiper 216 as the brushroll 26 rotates). With reference to FIG. 6, in another embodiment, the wiper 216 is an extension of the lens 208. Therefore, the wiper 216 may be made of the same material as the lens 208. In the embodiment of FIG. 6, the light source 70 is a UV bulb.

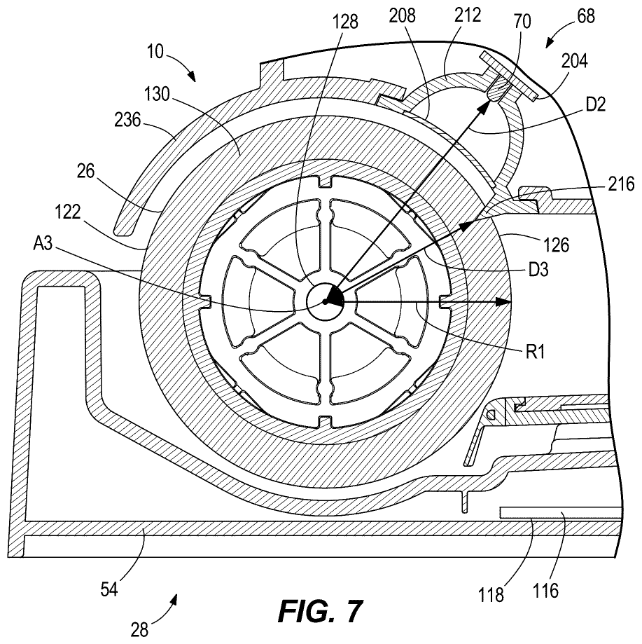

With reference to FIG. 7, in another embodiment, the floor cleaner 10 includes the light source 70 mounted in the brushroll housing 236. The brushroll housing 236 defines a brushroll housing axis that is parallel or collinear with the longitudinal axis A3 of the brushroll 26. As illustrated in the embodiment of FIG. 7, the floor cleaner 10 includes the wiper 216, which is configured to interfere with (i.e., engage) the brushroll 26 when the brushroll 26 rotates. The wiper 216 may be an extension of the brushroll housing 236 or an extension of the reflector 212. An operation of the light source 70 may be controlled by the control circuitry 40 onboard the floor cleaner (FIG. 2) or by the control circuitry 116 on the cleaning apparatus 28.

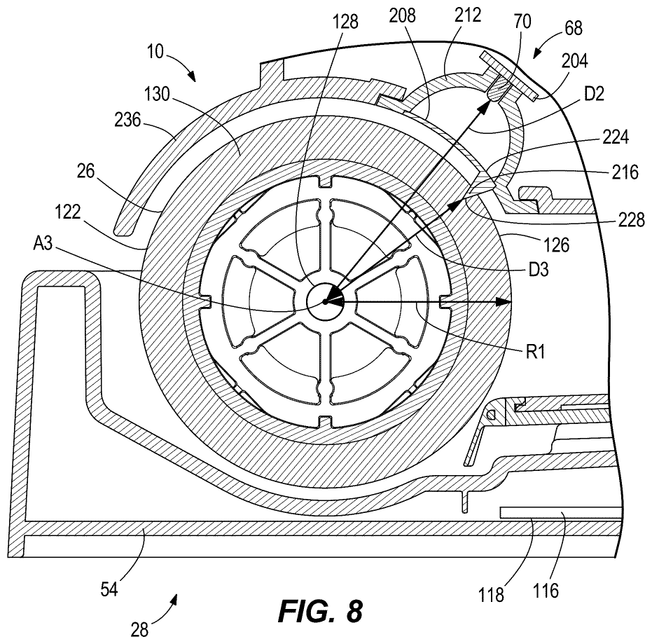

With reference to FIG. 8, in some embodiments, floor cleaner 10 includes the wiper 216, and the wiper 216 is an extension of the lens 208. As disclosed elsewhere herein, the wiper 216 deflects the operational material 130 on the brushroll 26 to expose a portion of an interior of the brushroll 26 to the UV light.

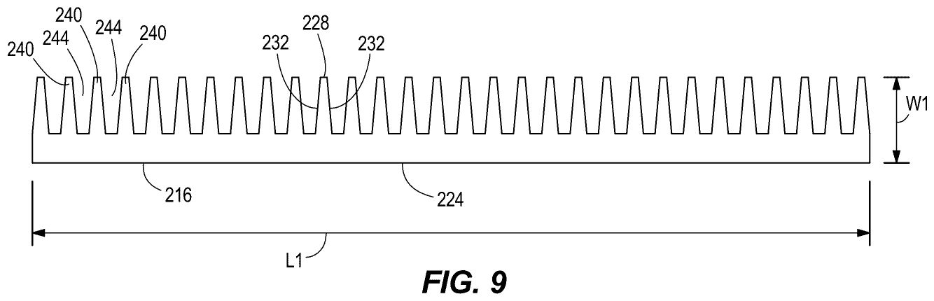

With reference to FIG. 9, the wiper 216 may have various shapes as desired. For example, the wiper 216 of FIG. 9 has a length L1 which, in some embodiments, is greater than 50% of a length of the brushroll 26, greater than 60% of the length of the brushroll 26, greater than 70% of the length of the brushroll 26, greater than 80% of the length of the brushroll 26, greater than 90% of a length of the brushroll 26, or substantially the same as a length of the brushroll 26. As such, the wiper 216 may interfere with (i.e., engage) the brushroll 26 along a substantial length of the brushroll 26. The wiper 216 has a width W1, and the width W1 may be sized to achieve a desired depth of interference (i.e., engagement, which is R1 minus D3) with the brushroll 26. In the embodiment of FIG. 9, the wiper 216 is shaped as a comb with a plurality of teeth 240. Spaces 244 are defined between the teeth 240 to allow the operational material 130 to pass between the teeth 240. The plurality of teeth 240 clean the brushroll 26 and remove dirt, trash, hair, and other debris from the brushroll 26 as the brushroll 26 rotates while engaged with the wiper 216. In some embodiments, the spaces 244 are omitted to provide an elongated wiper edge along the length L1. In other embodiments, the wiper edge of the wiper 216 is shaped as a squeegee or a scallop. The amount of engagement (i.e., R1 minus D3), the size of the spaces 244, and the length L1 are selected to provide the desired access of UV light to the interior of the brushroll 26 and such that the brushroll motor 46 that rotates the brushroll 26 operates within its load capacity.

With reference to FIG. 10, an embodiment of the cleaning apparatus 28 includes an air distributor system 246 including a fan 248 to deliver an airflow (indicated by the arrow 252) to the brushroll 26. Various versions of air distributor systems 246 are described herein. In the embodiment illustrated in FIG. 10, an air distributor 162 includes a plenum 114, e.g., a duct, leading from the fan 248 to one or more air openings 102. A first wall 134 and a second wall 138 are both located on the tray 54 and at least partially define the air opening 102. The walls 134, 138 also form a portion of the plenum 114. In some embodiments, the plenum 114 is a separate duct installed into the tray in fluid communication with the air opening 102. When the brushroll 26 is in the cleaning apparatus 28, the air opening 102 is disposed at a distance D4 from the longitudinal axis A3 of the brushroll 26 that is less than the radius R1 such that the air opening 102 interferes with the brushroll 26 to deflect the operational material 130, for example to deflect or separate fibers or bristles of the brushroll 26, and thereby dispense air into the brushroll 26 when the brushroll 26 is received within the brushroll-receiving portion 62 (e.g., when the base 12 is in the cleaning apparatus 28). In the illustrated embodiment, the air opening 102 is provided adjacent the intersection of the first and second walls 134, 138. In one cleaning mode, the brushroll 26 rotates as air is delivered from the air distributor 162, for example, to facilitate drying of the brushroll 26.

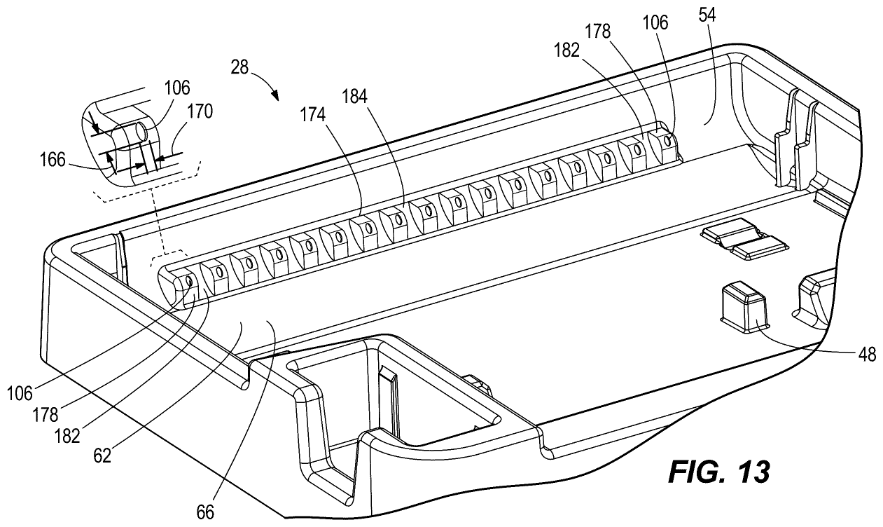

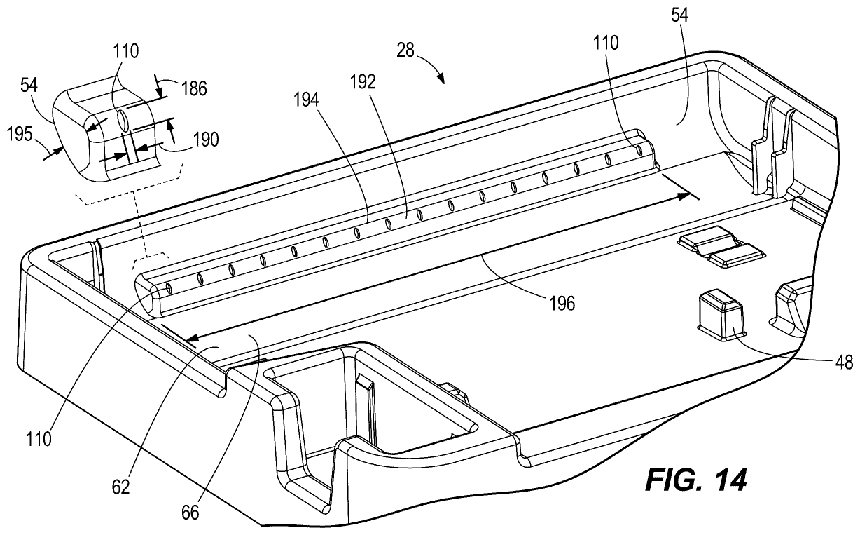

In some embodiments, air openings 102, 106, 110 of air distributors 162 (FIG. 12), 174 (FIG. 13), 194 (FIG. 14) are positioned offset from the brushroll 26 to not contact the brushroll 26. In other embodiments, the air openings 102, 106, 110 are positioned to interfere with (i.e., engage) the brushroll 26.

With continued reference to FIG. 10, in addition to or in place of the walls 134, 138, projections or ribs (not shown) may be located on the distributor 162 or tray 54 to interfere with the brushroll 26 to further clean the brushroll 26 (e.g. wipe, scrape, comb and/or separate bristles or fibers of the brushroll 26) when the brushroll 26 is in the cleaning apparatus 28 and is rotated. When the distance D4 is less than R1, the brushroll 26 engages the distributor 162 when the brushroll 26 rotates. When the operational material 130 of the brushroll 26 includes bristles or fiber, interference with the distributor 162 or other projections aids in separating the fibers as the brushroll 26 rotates and air is distributed from the air opening 102 (or, in other embodiments, from the air opening 106 (FIG. 13) or the air opening 110 (FIG. 14)), exposing more of the brushroll 26 to the air and facilitating improved drying of the brushroll 26. Stated another way, in some embodiments, the cleaning apparatus 28 is configured to engage the brushroll 26 and expose an internal portion of the brushroll 26 to the air openings 102, 106, 110, and the internal portion of the brushroll 26 includes portions of the brushroll 26 disposed at a dimension less than R1 from the rotational axis A3.

With continued reference to FIG. 10, the plenum 114 includes plenum walls 142. One or more of the plenum walls 142 may be molded into the tray 54 or otherwise integrated into the tray 54. In some embodiments, the plenum walls 142 form a plenum unit 146 that is removable from the tray 54 for purposes that may include, for example, cleaning.

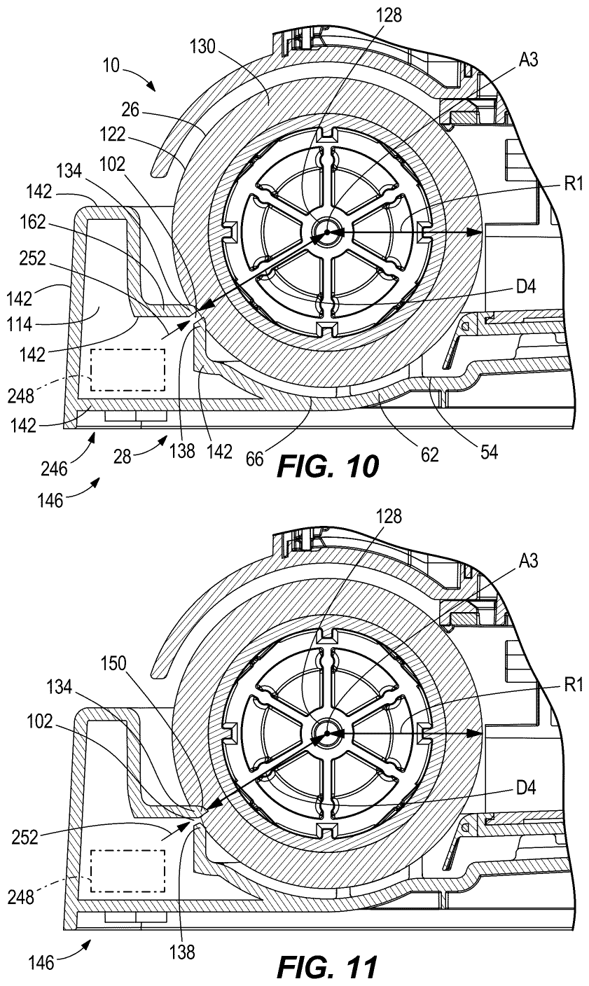

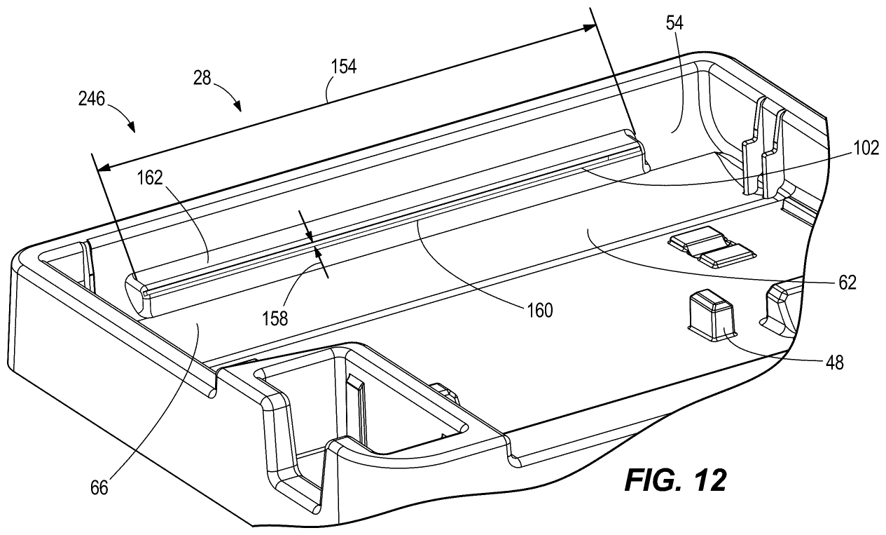

With reference to FIG. 11, an embodiment of a cleaning apparatus 28 as shown in FIG. 11 includes some features similar to the embodiment of the cleaning apparatus 28 shown in FIG. 10 and may include a cover portion 150 that is located on or adjacent to the first wall 134 or the second wall 138. The cover portion 150 shields at least a portion of the air openings 102, 106, 110 to inhibit an ingress of fluid and/or debris into the air openings 102, 106, 110. In some embodiments, the cover portion 150 interferes with the brushroll 26. The cover portion 150 may curve in a direction over the air opening 102, 106, 110.

With reference to FIG. 12, the air distributor 162 includes the air opening 102 positioned along the brushroll-receiving portion 62. The air opening 102 may be dimensioned as a slot having a major dimension 154 and a minor dimension 158. The major dimension 154 and the minor dimension 158 may be different lengths as desired to achieve the desired opening area and air flow rate. The major dimension 154 extends along the brushroll 26 when the brushroll 26 is in the brushroll-receiving portion 62. In the illustrated embodiment, the air opening 102 extends along a majority of the brushroll-receiving portion 62. The air opening 102 may extend along various lengths of the brushroll-receiving portion 62 as disclosed herein. In one embodiment, the air distributor 162 includes a longitudinal ridge 160 and the air opening 102 extends along the longitudinal ridge 160. The air opening 102 may be positioned on the distributor 162 and in communication with the plenum 114 such that air can pass through the plenum 114 and pass through the air opening 102. The distributor 162 may extend along the longitudinal axis A2 (FIG. 3) of the brushroll-receiving portion 62 and may be positioned to direct air toward the brushroll 26 when the floor cleaner 10 is in the mounted position.

With reference to FIG. 13, in another embodiment of the cleaning apparatus 28, a plurality of air openings 106 extend along an air distributor 174 and are adjacent the brushroll-receiving portion 62. The air distributor 174 includes a plurality of outwardly extending segments 178 disposed along the longitudinal axis A2 and/or longitudinal axis A3. At least one of the plurality of segments 178 includes at least one of the plurality of air openings 106. In the illustrated embodiment, each of the plurality of segments 178 includes one air opening 106. However, in other embodiments the segments 178 and the air openings 106 are arranged as desired to achieve the desired airflow distribution along the brushroll. Each air opening 106 may be dimensioned as a port, ellipse, circle, and/or jet having a major dimension 166 and a minor dimension 170. The major dimension 166 may be positioned at a right angle to the minor dimension 170. In some embodiments, the major dimension 166 is the same as the minor dimension 170 (for example, when the air opening 106 is a circle or a square). Each air opening 106 may be any size or shape with a major dimension 166 and a minor dimension 170 that are sized as desired to achieve the desired opening area and air flow rate. In the embodiment of FIG. 13, the air openings 106 are positioned on the outwardly extending projections or segments 178 along the distributor 174 in communication with the plenum 114 such that air can pass through the plenum 114 and pass through the segments 178 to the air openings 106. The distributor 174 may extend in a direction parallel to or generally parallel to the longitudinal axis A2 of the brushroll-receiving portion 62. The segments 178 may be provided in the brushroll-receiving portion 62 to engage the brushroll 26. In one embodiment, the segments 178 include an arcuate shape. In other embodiments, the segments 178 may be shaped as a cone or a truncated conc. In some embodiments, the cone-shaped segments 178 may point toward the longitudinal axis A2 of the brushroll-receiving portion 62. Each segment 178 has a width or thickness dimension along the longitudinal axis A2 that may be selected as desired to engage the brushroll 26. In some embodiments, the width dimension of the segments 178 may be narrow to form a comb shape (e.g., a shape that combs debris that may include hair from the brushroll 26) such as that illustrated by the wiper 216 in FIG. 9. The dimension D4, representing the distance between the longitudinal axis A3 of the brushroll 26 (and/or the longitudinal axis A2 of the brushroll-receiving portion 62) and the air openings 102, 106, 110 may be selected to provide desired air delivery and to reduce friction between the brushroll 26 operational material 130 and the distributors 162, 174, 194 such that the brushroll motor 46 that rotates the brushroll 26 operates within its load capacity. Further, the segment width or thickness dimension along the longitudinal axis A3 (and/or along the longitudinal axis A2) may be selected to provide desired air delivery and reduce friction between the brushroll 26 operational material and the distributor 174 such that the brushroll motor 46 that rotates the brushroll 26 operates within its load capacity. In the illustrated embodiment, the distributor 174 includes a plurality of segments 178 that function similarly to the walls 134, 138 described herein to clean the brushroll 26 by interfering with the brushroll 26 when the brushroll 26 is placed in the brushroll-receiving portion 62 and rotated (that is, activated) and to dispense air through the air openings 106 into the brushroll 26. The air that is dispensed within the outer perimeter 126 of the brushroll 26 through the air openings 102, 106, 110 dries (i.e., by facilitating the evaporation of moisture) the operational material 130 of the brushroll 26, particularly operational material 130 that is positioned inward of the outer perimeter 126 of the brushroll 26, which may otherwise be difficult to dry by merely dispensing air onto the outer perimeter 126 of the brushroll 26. Therefore, dispensing air into an interior of the brushroll 26, and more specifically onto the operational material 130 inside the outer perimeter 126, facilitates improved drying of the operational material 130.

With continued reference to FIG. 13, a gap or divot 182 is formed between adjacent segments 178. Stated another way, each segment 178 is separated from an adjacent segment 178 by one of the divots 182. With analogous reference to FIG. 10, the distance D4 may be similarly defined in the embodiment of FIG. 13 as a radial distance between the longitudinal axis A3 of the brushroll 26 and/or the longitudinal axis A2 of the brushroll-receiving portion 62 to one or more of the air openings 106. In some embodiments, one or more of the divots 182 may be positioned a radial third distance from the longitudinal axis A2 of the brushroll-receiving portion 62, the third distance being measured to a rear wall 184 bounding the divots 182 and being greater than the second distance D2. In the illustrated embodiment, seventeen air openings 106, seventeen segments 178, and sixteen divots 182 are provided. However, the number of air openings 106 and segments 178 may be increased or decreased as desired to provide desired air delivery to the brushroll 26 for the application.

With reference to FIG. 14, in another embodiment of the cleaning apparatus 28, the air distributor 194 includes an outwardly extending longitudinal ridge 192 extending along at least a portion of the brushroll-receiving portion 62. A plurality of air openings 110 extend along the distributor 194 and adjacent the brushroll-receiving portion 62. At least one of the plurality of air openings 110 extend along the longitudinal ridge 192. In the illustrated embodiment, the plurality of air openings 110 extend along the longitudinal ridge 192. However, in other embodiments the longitudinal ridge 192 and the air openings 110 are arranged as desired to achieve the desired airflow distribution along the brushroll 26. Each air opening 110 may be dimensioned as a port, ellipse, circle, and/or jet having a major dimension 186 and a minor dimension 190. The major dimension 186 may be positioned at a right angle to the minor dimension 190. In some embodiments, the major dimension 186 may be the same as the minor dimension 190 (for example, when the air opening 110 is a circle or a square). Each air opening 110 may be any size or shape with a major dimension 186 and a minor dimension 190 that are sized as desired to achieve the desired opening area and air flow rate. In the embodiment of FIG. 14, the air openings 110 are positioned on the outwardly extending longitudinal ridge 192 along the distributor 194 in communication with the plenum 114 such that air can pass through the plenum 114 to the air openings 110. In the illustrated embodiment, the longitudinal ridge 192 has a height 195 that is measured normal to a portion of the tray 54 from which the longitudinal ridge 192 extends and a length 196. The height 195 is selected to position the air openings 110 at their desired positions relative to the brushroll 26 and, in general, may be sized as desired. The distributor 194 may extend in a direction parallel to or generally parallel to the longitudinal axis A2 (FIG. 3) of the brushroll-receiving portion 62. In the illustrated embodiment, the air openings 110 are equidistantly spaced apart, and further are equidistantly spaced apart on the distributor 194. In the illustrated embodiment, the distributor 194 functions similarly to the walls 134, 138 described herein to dispense air into the brushroll 26, e.g. onto the operational material 130 inside the outer perimeter 126, by interfering with the brushroll 26 when the brushroll 26 is placed in the brushroll-receiving portion 62 and rotated (that is, activated).

With continued reference to FIG. 14, and with analogous reference to FIG. 10, the distance D4 may be similarly defined in the embodiment of FIG. 14 as a radial distance between the longitudinal axis A3 of the brushroll 26 and/or a radial distance between the longitudinal axis A2 of the brushroll-receiving portion 62 to one or more of the air openings 110.

With returning reference to FIGS. 1 and 2, the floor cleaner 10 includes control circuitry 40 including a controller. The control circuitry 40 is configured to control operation of one or more components of the floor cleaner 10. The control circuitry 40 is operably connected to the brushroll motor 46, the suction motor 20, and the fluid distributor 56 and is configured to operate the floor cleaner 10 upon user actuation. The brushroll 26 is driven by the brushroll motor 46, and the control circuitry 40 is connected to and operable to control the brushroll motor 46. The control circuitry 40 is operable in a brushroll-enabled condition when the floor cleaner 10 is in the inclined use position or other operational condition; a brushroll-disabled condition when the floor cleaner 10 is in the upright position or other storage condition and the floor cleaner 10 is not in the cleaning apparatus 28; and a brushroll-enabled condition when the floor cleaner 10 is in the mounted position. Thus, when the floor cleaner 10 is in the upright storage position, the brushroll 26 will not be activated unless the floor cleaner 10 is in the cleaning apparatus 28.

In some embodiments, the controller of the control circuitry 40 is electrically and/or communicatively connected to a variety of modules or components of the floor cleaner 10. The controller includes a plurality of electrical and electronic components that provide power, operational control, and protection to the components and modules within the controller and/or the floor cleaner 10. The controller further includes an electronic processor (for example, a microprocessor or another suitable programmable device) and memory. The memory includes, for example, a program storage area and a data storage area. The program storage area and the data storage area can include combinations of different types of memory, such as read-only memory (ROM), random access memory (RAM). Various non-transitory computer readable media, for example, magnetic, optical, physical, or electronic memory may be used. The electronic processor is communicatively coupled to the memory and executes software instructions that are stored in the memory, or stored on another non-transitory computer readable medium such as another memory or a disc. The software may include one or more applications, program data, filters, rules, one or more program modules, and other executable instructions. The controller receives a signal(s) indicative of a characteristic(s) and controls the floor cleaner 10 accordingly (for example, by varying a control signal). Although the controller is described as one controller, the controller could also include multiple controllers configured to work together to achieve a desired level of control for the floor cleaner 10. As such, any control functions and processes described herein with respect to the controller could also be performed by two or more controllers functioning in a distributed manner.

With continued reference to FIG. 2, in one embodiment, the control circuitry 40 includes two switches 42, 44. The first switch 42 indicates when the floor cleaner 10 is in the upright storage position. The second switch 44 indicates when the floor cleaner 10 is in the mounted position in the cleaning apparatus 28. The first switch 42 and second switch 44 may be mechanical or electrical switches, and may be activated by sensor input, by mechanical actuation, or other switching techniques. In some embodiments having a floor cleaner 10 without an upright storage position, the first switch 42 is omitted.

With reference to FIGS. 2 and 3, the second switch 44 may be positioned in the floor cleaner 10, for example, in the base 12, configured to be activated by an actuator 48 in the cleaning apparatus 28. The location of the second switch 44 and the location of the actuator 48 are selected such that the actuator 48 activates the second switch 44 when the floor cleaner 10 is in the mounted position, and the actuator 48 does not activate the second switch 44 when the floor cleaner 10 is not in the mounted position. The actuator 48 and second switch 44 may be positioned to ensure the floor cleaner 10 is in a desired position and alignment relative to features of the cleaning apparatus 28. The actuator 48 enables the brushroll 26 when the base 12 of the floor cleaner 10 is received in the tray 54 in that the actuator 48 is configured to activate the second switch 44 signaling to the control circuitry 40 that the floor cleaner 10 is in the mounted position, and the control circuitry 40 puts the brushroll 26 in the brushroll-enabled condition. In one embodiment, the second switch 44 is a mechanically actuated switch (for example, a contact switch), and the actuator 48 is a mechanical actuator 48 positioned in the cleaning apparatus 28. The mechanical actuator 48 in the cleaning apparatus 28 may be a rib, a pin, a protrusion, or other shape or feature positioned to actuate the second switch 44 in the floor cleaner 10 when the floor cleaner 10 is placed in the mounted position in the cleaning apparatus 28. In the example shown in FIG. 3, the mechanical actuator 48 is a protrusion extending upwardly positioned to engage the second switch 44 in the base 12. The second switch 44 may be adjacent an outer surface of the base 12 and positioned for direct engagement with the mechanical actuator 48. In one embodiment, the mechanical actuator 48 enters a recess or aperture in the base 12 to actuate the second switch 44. In one embodiment, the actuator 48 contacts a linkage or other intermediate member that actuates the second switch 44 in the floor cleaner 10 when the base 12 is placed in the cleaning apparatus 28.

In one embodiment, the second switch 44 is a magnetically activated switch (for example, a reed switch or a switch using a Hall Effect sensor), and the actuator 48 is a magnet. In this embodiment, the reed switch is positioned in the floor cleaner 10 in a location corresponding to the location of the magnet in the cleaning apparatus 28 when the floor cleaner 10 is placed in the mounted position in the cleaning apparatus 28. In another embodiment, the second switch 44 includes a Hall Effect sensor and the actuator 48 is a magnet positioned in the cleaning apparatus 28.

In one embodiment, the second switch 44 is a light activated switch (for example, a switch using a photodetector or photoresistor or a switch using a proximity sensor), and the actuator on the cleaning apparatus 28 is a light, a shape, a bar code, a reflector, or other feature sensed by the light activated switch when the floor cleaner 10 is placed in the mounted position in the cleaning apparatus 28. In other embodiments, the second switch 44 may be another type of proximity sensor configured to cooperate with the cleaning apparatus 28 to actuate the control circuitry 40, such as a pressure sensor, ultrasonic sensor, or other proximity sensor.

In the illustrated embodiment, the actuator 48 includes electrical contacts 60 (FIG. 3) and the second switch 44 is an electronic switch operatively connected to electrical contacts positioned to electrically connect with the actuator electrical contacts 60 when the floor cleaner 10 is in the mounted position, and the controller is configured to determine when the actuator 48 has connected to the second switch 44, for example by sensing a change in resistance or voltage across the second switch 44, or receiving a signal from the cleaning apparatus control circuitry 116, or other sensing technique.

With returning reference to FIGS. 1 and 2, the floor cleaner 10 and the cleaning apparatus 28 may be operated in a plurality of various cleaning modes. For example, in one cleaning mode, the floor cleaner 10 operates a self-cleaning cycle while the ultraviolet light generator 68 is not operating. In another cleaning mode, the floor cleaner 10 operates a self-cleaning cycle while the ultraviolet light generator 68 is operating. In another cleaning mode, the cleaning apparatus 28 operates a cleaning cycle while the floor cleaner 10 operates portions of the floor cleaner 10 in cooperation with the cleaning cycle, for example, by operating the brushroll motor 46 during all or a portion of the cleaning cycle. In some cleaning cycles, the air distributor system 246 (shown in FIGS. 10 and 12, for example) and the ultraviolet light generator 68 (FIGS. 3-8) may operate simultaneously or sequentially (for example, in one embodiment, the floor cleaner 10 operates a self-cleaning cycle, and then the air distributor system 246 operates, and then the ultraviolet light generator 68 operates, or the systems or cycles operate in another sequence as desired). In another cleaning mode, the floor cleaner 10 operates the brushroll motor 46 while the air distributor system 246 is operating a drying cycle.

The floor cleaner 10 may be operable in a cleaning mode having a self-cleaning cycle in which the controller is configured to control operation of one or more components of the floor cleaner 10 to clean one or more portions of the floor cleaner 10, for example the suction inlet 30 and the brushroll 26. The cleaning mode may be configured to operate when the floor cleaner 10 is in the mounted position in the cleaning apparatus 28. In one embodiment, the second switch 44 is configured to provide a signal to the controller when the second switch 44 is activated by the actuator 48 such that the control circuitry 40 senses the cleaning apparatus 28. In some embodiments, the second switch 44 enables the cleaning mode by the controller being configured to not operate the self-cleaning cycle of the cleaning mode unless the second switch 44 indicates that the floor cleaner 10 is in the mounted position. The controller is operatively connected to the fluid distributor 56 and may be configured to distribute solution from the supply tank 16 in response to activation of the second switch 44 by the cleaning apparatus 28 and initiation of the cleaning mode having the self-cleaning cycle. The controller is operatively connected to the suction motor 20 and configured to activate the suction motor 20 in response to activation of the second switch 44 by the cleaning apparatus 28 and initiation of a cleaning mode having a cleaning cycle. In some embodiments, the floor cleaner 10 is operable in a cleaning mode having a self-cleaning cycle when the floor cleaner 10 is not in the mounted position in the cleaning apparatus 28. Rather, for example, the floor cleaner 10 may be operable in a cleaning mode having a self-cleaning cycle when the floor cleaner 10 is scrubbing a surface to be cleaned.

In one cleaning mode, when the floor cleaner 10 is in the cleaning apparatus 28, the controller receives a signal from the second switch 44 indicating that the floor cleaner 10 is in the mounted position in the cleaning apparatus 28, thereby enabling the cleaning mode. After actuation of the cleaning mode having a cleaning cycle, the controller operates the fluid distributor 56 to dispense cleaning fluid through the distribution nozzle 32 to the tray 54 of the cleaning apparatus 28. Subsequently, the controller activates the brushroll motor 46 to rotate the brushroll 26 in the cleaning fluid disposed in the tray 54. The controller further activates the suction motor 20 to extract the cleaning fluid from the brushroll 26 and the tray 54. The air distributor system 246 may be activated during the cleaning cycle and, more particularly in some embodiments, after the extraction phase of the cleaning cycle, to facilitate drying of the brushroll 26. In some embodiments of a cleaning cycle, the ultraviolet light generator 68 is operated to further clean or sanitize, and in one example to inhibit microbial growth on and/or within, the brushroll 26. In some embodiments, the cleaning cycle of the cleaning mode is prevented when the floor cleaner 10 is not in the mounted position in the cleaning apparatus 28.

The ultraviolet light generator 68 may be controlled to interrupt operation when the second switch 44 indicates that the floor cleaner 10 is not in the mounted position to limit UV exposure to people and animals. In some embodiments, the controller is configured to not operate the ultraviolet light generator 68 unless the second switch 44 indicates that the floor cleaner 10 is in the mounted position, and to interrupt operation of the ultraviolet light generator 68 if the second switch 44 indicates that the floor cleaner 10 is removed from the mounted position during operation of the cleaning cycle.

The cleaning apparatus 28 is configured to at least partially surround the distribution nozzle 32, such that cleaning fluid from the distribution nozzle 32 contacts the cleaning apparatus 28 and is directed into the tray 54. The fluid distributor 56, shown schematically in FIG. 1, may include the auxiliary fluid outlet 36 in communication with the supply tank 16. The auxiliary fluid outlet 36 may be controlled by a valve 38 activated by the second switch 44. The auxiliary fluid outlet 36 may be configured to provide a flow rate greater than that provided by the pump 34 and distribution nozzle 32. The auxiliary fluid outlet 36 is positioned above the tray 54 when the base 12 is positioned on the cleaning apparatus 28 such that cleaning fluid from the supply tank 16 flows into the tray 54 when the valve 38 is actuated by the second switch 44.

In one embodiment, a cleaning cycle of a cleaning mode is initiated manually by the user operating the floor cleaner 10 while the floor cleaner 10 is in the mounted position in the cleaning apparatus 28. For example, a cleaning cycle of a cleaning mode may be initiated when the user turns the floor cleaner 10 on by depressing, for example, a pushbutton 50, and then the user operates the floor cleaner 10 to dispense cleaning fluid into the tray 54 by activating a trigger 52, which in the illustrated embodiment is located on the handle of the floor cleaner 10, when the floor cleaner 10 is in the cleaning apparatus 28. The pushbutton 50, which is an on/off button in one example, activates the suction motor 20 and the brushroll motor 46, while the trigger 52 distributes cleaning fluid to the cleaning apparatus 28. The brushroll 26 rotates in the cleaning fluid collected in the tray 54 to remove debris from the brushroll 26, whereupon the cleaning fluid and debris are sucked through the suction inlet 30 and through the recovery path to the recovery tank 18 of the floor cleaner 10.

In one embodiment, a cleaning cycle of a cleaning mode operates automatically after the cleaning cycle is initiated. An automated cleaning cycle is controlled by the control circuitry 40 including the floor cleaner controller. The controller is operatively connected to the fluid distributor 56, the suction motor 20, and the brushroll motor 46. The controller is further operably coupled with a user interface for receiving inputs from the user. In response to user activation or other activation, the controller is configured to activate an automated cleaning cycle of a cleaning mode when the floor cleaner 10 is in the cleaning apparatus 28. The controller operates the suction motor 20, the brushroll motor 46, and the fluid distributor 56 in the cleaning cycle.

A cleaning cycle may include, and in some embodiments may consist of, a distribution phase and an extraction phase. During the distribution phase, cleaning fluid is distributed from the supply tank 16 through the fluid distributor 56, and into the tray 54 of the cleaning apparatus 28. In one embodiment, cleaning fluid is placed into the tray by the user prior to initiating the cleaning cycle. During the extraction phase, the brushroll motor 46 is activated, allowing the brushroll 26 to rotate in the cleaning fluid in the cleaning apparatus 28 to remove dirt and debris. The suction motor 20 is also activated to extract the cleaning fluid, dirt, and debris through the suction inlet 30. The distribution phase and extraction phase may run simultaneously or may be staggered or sequential. In one embodiment, a cleaning cycle includes a distribution phase, an agitation phase, and an extraction phase. In this embodiment, cleaning fluid is distributed from the supply tank 16 into the tray 54 in the distribution phase. In the agitation phase, the brushroll motor 46 is activated, rotating the brushroll 26 in the cleaning fluid in the cleaning apparatus 28 to remove dirt and debris while the suction motor 20 is not activated or activated at a reduced power level. Then, the suction motor 20 is activated in the extraction phase. The distribution phase, agitation phase, and extraction phase may run simultaneously, or may be staggered or sequential. A cleaning cycle can optionally repeat the distribution phase and/or extraction phase and/or agitation phase if present one or more times. The length of each phase and quantity of cleaning fluid dispensed in a cleaning cycle may be time-dependent or may continue until the recovery tank 18 is full or the supply tank 16 is empty. In one embodiment, the controller operates the suction motor 20, the brushroll motor 46, and the fluid distributor 56 based on another control scheme, such as monitoring cleaning effectiveness by sensors.

In one embodiment of an automated cleaning cycle, a user activation is the user actuating a pushbutton 50 on the floor cleaner 10 or the cleaning apparatus 28. In another embodiment, a user activation is placing the floor cleaner 10 into the cleaning apparatus 28. In yet another embodiment, a user activation is by an infrared, Wi-Fi, Bluetooth, or other signal sent from a remote device by user interaction with the device, such as a remote control, cell phone, or computer.

During an ultraviolet light cleaning cycle, which may be activated automatically or manually, the cleaning apparatus 28 and/or the floor cleaner 10 activates the light source 70 to deliver ultraviolet light to the brushroll 26. In some embodiments, the light source 70 may be activated only if the floor cleaner 10 is in the mounted position in the cleaning apparatus 28. In other embodiments, such as, for example, embodiments in which the floor cleaner 10 includes the light source 70, the light source 70 may be activated with the floor cleaner 10 in the mounted position in the cleaning apparatus 28 and/or with the floor cleaner 10 not in the mounted position. More generally, the ultraviolet light generator 68 is activated during a cleaning cycle to at least partially clean, sanitize, or enhance the brushroll 26. More specifically, and in some embodiments of an ultraviolet light cleaning cycle of a cleaning mode, the brushroll 26 rotates while ultraviolet light is directed into the brushroll 26. The intensity and duration of the light delivered to the brushroll 26 may be selected to provide a desired cleaning effect. Further, the rotational speed of the brushroll 26 and the temporal length of the ultraviolet light cleaning cycle may be selected to provide a desired cleaning effect. The speed of the brushroll rotation may be less than 1 degree/second, or between 1 and 10 degrees per second, or any other rotational speed as desired to provide a dwell time for the ultraviolet light to contact the brushroll 26. The light source 70, the position of the light source 70 including the distance of the light source 70 from the brushroll 26 or other components, the wavelength of the light, and the rotational speed of the brushroll 26 are selected collectively to provide a desired strength, or dose, of UV light to provide a desired level of cleaning, for example, between 10 and 130, or more, Joules per square meter, as desired for the application. In one application, for example, a low strength, or dose, may be desired for freshening the brushroll, or providing a visual lighting effect, such as between 10 and 60 Joules per square meter. However, in some applications, a higher strength may be used, such as between 60 and 130 Joules per square meter, for example, for some sanitizing and disinfecting applications.

In some embodiments, the floor cleaner 10 and the cleaning apparatus 28 are configured to communicate when the floor cleaner 10 is in the mounted position. In the illustrated embodiment, the cleaning apparatus control circuitry 116, including the PCBA 118, is electrically connected to the actuator electrical contacts 60 and configured to send and receive signals through the electrical contacts 60 or through other electrical contacts when the floor cleaner 10 is in electrical connection with the actuator 48 in the mounted position. In one embodiment, the floor cleaner controller is configured to instruct the cleaning apparatus control circuitry 116 to operate the ultraviolet light generator 68 in an ultraviolet light cleaning cycle of a cleaning mode. In some examples, the cleaning apparatus control circuitry 116 is configured to operate the ultraviolet light generator 68 in an ultraviolet light cleaning cycle of a cleaning mode. The ultraviolet light cleaning cycle includes an ultraviolet light distribution phase in which ultraviolet light is directed toward the brushroll 26 and/or within the outer perimeter 126 of the brushroll 26. The ultraviolet light may be reflected toward the brushroll 26 by the reflector 212 and may pass through the lens 208. In a brushroll activation phase, the brushroll 26 rotates to allow the ultraviolet light to contact a circumference of the brushroll 26 and to allow for a desired dwell time of the ultraviolet light at particular locations on the brushroll 26. The ultraviolet light distribution phase may occur simultaneously with the brushroll activation phase.

In one embodiment, the cleaning apparatus control circuitry 116 senses whether the floor cleaner 10 is in the mounted position, for example by sensing a change in resistance or voltage across the actuator electrical contacts 60, receiving a signal from the floor cleaner control circuitry 40, or other sensing technique. In one embodiment, a third switch is provided on the cleaning apparatus 28 and actuated by the floor cleaner 10 when the floor cleaner 10 is in the mounted position. In some embodiments, the cleaning apparatus control circuitry 116 prevents operation of the ultraviolet light generator 68 when the floor cleaner 10 is not in the mounted position.

A cleaning cycle and an ultraviolet light cleaning cycle of a cleaning mode may be operable such that, in some embodiments, a cleaning cycle and an ultraviolet light cleaning cycle may be operated individually, sequentially, or simultaneously. In other words, in an embodiment, a first cleaning cycle of a cleaning mode, in which ultraviolet light is not emitted, may be operated prior to operating an ultraviolet light cleaning cycle of the cleaning mode, in which the cleaning apparatus 28 and/or the floor cleaner 10 emits ultraviolet light onto and/or into the brushroll 26. In another embodiment, a single cleaning cycle of a cleaning mode may run that combines ultraviolet light emission with a cleaning cycle. A user may actuate a single switch to activate both a cleaning cycle and an ultraviolet light cleaning cycle of a cleaning mode, or in some embodiments, a user may actuate separate switches to activate a cleaning cycle and an ultraviolet light cleaning cycle of a cleaning mode individually or sequentially.

The floor cleaner 10 may be cleaned in one embodiment by a method of cleaning a floor cleaner 10 using a cleaning apparatus 28 having an actuator 48 by receiving the floor cleaner 10 in the mounted position in the cleaning apparatus 28. The method includes sensing the presence of the cleaning apparatus 28 by control circuitry 40 in the floor cleaner 10 that is activated by the actuator 48. Stated another way, the method may include activating the control circuitry 40 in the floor cleaner 10 by the cleaning apparatus actuator 48. The method further comprises activating a cleaning mode that distributes cleaning fluid from a supply tank 16 into the cleaning apparatus 28. In a further embodiment, the brushroll 26 may be activated in a cleaning mode when the floor cleaner 10 is in the mounted position. A method may further comprise activating the suction motor 20 to extract cleaning fluid from the cleaning apparatus 28. In one embodiment, the control circuitry 40 may be configured to prevent the activation of or to interrupt a cleaning mode if the cleaning apparatus 28 is not sensed or if the floor cleaner 10 is not in the upright position. The method may further comprise, in a cleaning mode, distributing cleaning fluid from the supply tank 16 into the tray 54 via the fluid distributor 56, the supply tank 16 being on the floor cleaner 10. The method may be such that the floor cleaner 10 includes a suction motor 20, the method further comprising extracting the cleaning fluid from the tray 54 and into the recovery tank 18 on the floor cleaner 10 by activating the suction motor 20. The method may be such that the step of activating a cleaning mode includes activating a cleaning mode in response to a user activation. The method may further comprise activating the brushroll 26 while ultraviolet light is being directed toward the brushroll 26 and/or into the brushroll 26 to provide a desired dwell time of the ultraviolet light on the operating material 130.

The floor cleaner 10 may be cleaned in one embodiment by a method of ultraviolet light cleaning of a floor cleaner 10 using a cleaning apparatus 28 having an actuator 48 by receiving the floor cleaner 10 in the mounted position in the cleaning apparatus 28. The method includes sensing the presence of the cleaning apparatus 28 by a control circuitry 40 in the floor cleaner 10 that is activated by the actuator 48. Stated another way, the method may include activating the control circuitry 40 in the floor cleaner 10 by the actuator 48. The method may further include emitting ultraviolet light onto the brushroll 26 while the brushroll 26 is positioned in the cleaning apparatus 28. The method may further comprise directing the ultraviolet light towards the brushroll 26. The method may further comprise wiping the brushroll 26 to expose an interior of the brushroll 26 to ultraviolet light. The method may further comprise providing ultraviolet light into an interior of the brushroll 26. The method may further comprise sensing that a portion of the base 12 of the floor cleaner 10 is received by the cleaning apparatus 28, and the sensing may be performed by the cleaning apparatus control circuitry 116 or the control circuitry 40 in the floor cleaner 10. The method may further comprise, when the control circuitry 40 senses that the portion of the base 12 of the floor cleaner 10 is received by the cleaning apparatus 28, activating a cleaning mode. The method may further comprise preventing activation of or interrupting a cleaning mode if the control circuitry 40 does not sense that the portion of the base 12 of the floor cleaner 10 is received by the cleaning apparatus 28. The method may be such that the step of activating a cleaning mode includes activating a cleaning mode in response to a user activation. The method may further comprise activating the brushroll 26 while the ultraviolet light is being directed toward and/or into the brushroll 26. The method may include activating the ultraviolet light and activating the brushroll 26 in desired intervals, such as rotating the brushroll 26 for a desired time or angular interval, and then stopping or slowing the brushroll 26 while the ultraviolet light is activated, and then resuming rotation of the brushroll 26 for a desired time or angular interval, and so on. In some embodiments, the ultraviolet light may also be activated in time intervals. In some embodiments, the ultraviolet light is activated in time intervals in coordination with rotation of the brushroll 26.

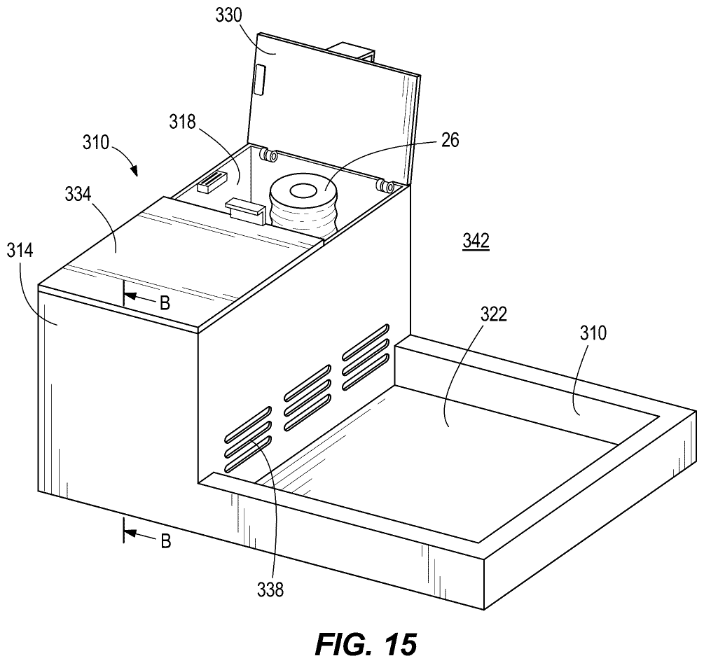

With reference to FIG. 15, a cleaning apparatus 310 is configured for cleaning a floor cleaner such as the floor cleaner 10. The cleaning apparatus 310 may include any one or more of the cleaning modes, methods, features, and embodiments described in this detailed description with respect to the cleaning apparatus 28 and the tray 54, individually or in desired combinations. The cleaning apparatus 310 includes a housing 314 defining a compartment 318 within the housing 314. In the illustrated embodiment, the cleaning apparatus 310 further includes a tray 322 configured for receiving the floor cleaner 10 in a mounted position in the tray 322. The tray 322 is at least partially bounded, and in the illustrated embodiment is bounded on three sides, by a wall system 326 that is configured to retain cleaning solution such as, for example, cleaning solution that is dispensed by the floor cleaner 10 during a cleaning cycle, in the tray 322. A fourth side of the tray 322 is bounded by the housing 314. In some embodiments, the cleaning apparatus 310 does not include the tray 322.

With continued reference to FIG. 15, the housing 314 includes a first door 330, which in some embodiments may be referred to as a first component door or as a brushroll door, and a second door 334, which in some embodiments may be referred to as a second component door or as a recovery tank door. Each of the doors 330, 334 is positionable in a closed position, in an open position, and in a range of positions therebetween to selectively provide access to the compartment 318. In the illustrated embodiment, the doors 330, 334 are hinged, are positioned adjacent to each other such that they open in opposite directions, and are provided on an upper portion or upper side 328 of the housing 314, but in other embodiments, the doors 330, 334 may be provided in other locations as desired. The upper side 328 is opposite a lower side 332 of the housing 314. In some embodiments, instead of an access door, all or a portion of the housing 314 is a cover that lifts from the tray 322 for access to the inside of the housing 314. Further, in some embodiments, only one door may be provided, and the door or doors 330, 334 may open in other manners, such as by sliding. In some embodiments, the door or doors 330, 334 may be latched in the closed position by a detent, catch, magnet, latch, or other closure as desired. One or more air vents 338 may be provided on the housing 314 to provide air communication between the compartment 318 and an outside environment 342. In the illustrated embodiment, the air vents 338 are positioned adjacent to the tray 322, but in other embodiments, the air vents 338 may be positioned as desired.

With returning reference to FIGS. 1 and 2, one or more components of the floor cleaner 10 such as the brushroll 26, the recovery tank 18, etc. is removable from the floor cleaner 10 for cleaning the one or more components of the floor cleaner 10. More specifically, the brushroll 26, the recovery tank 18, and/or other components of the floor cleaner 10 are selectively removable and are able to be cleaned and re-installed in the floor cleaner 10. When removed from the floor cleaner 10, one or more of the components of the floor cleaner 10 such as the brushroll 26, the recovery tank 18, and/or other components of the floor cleaner 10 are able to be placed in the compartment 318 of the cleaning apparatus 310 for cleaning.

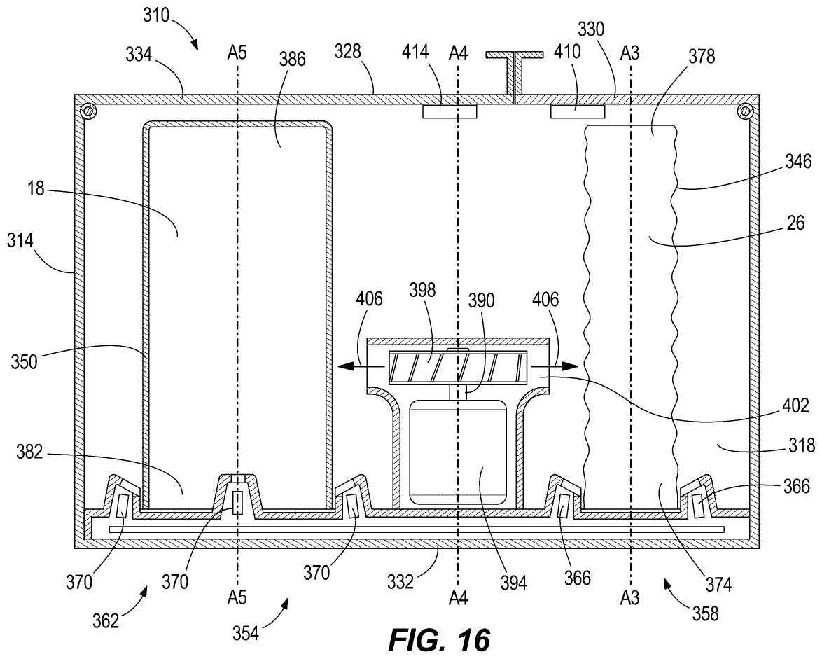

With reference to FIGS. 15 and 16, one or more removable components of the floor cleaner 10, such as the brushroll 26 and the recovery tank 18, are respectively placed in a brushroll-receiving location 346 and in a recovery tank-receiving location 350 in the compartment 318 of the cleaning apparatus 310. The cleaning apparatus 310 includes one or more cleaning members (e.g., a light, a fan, etc.) disposed within the compartment 318 and configured to facilitate cleaning and/or drying of the removable components of the floor cleaner 10. In some embodiments, the cleaning member is operable in at least one of a cleaning mode or a drying mode. For example, within the compartment 318, the brushroll 26 and the recovery tank 18 are exposed to a cleaning member delivering ultraviolet light that is emitted by an ultraviolet light generator 354 including one or more light sources and, more specifically in the illustrated embodiment, two light sources 358, 362 that are configured to emit ultraviolet light onto the components at which the light sources 358, 362 are directed. Each of the two light sources 358, 362 even more specifically may include a plurality of respective smaller light sources 366, 370. The first light source 358 may be referred to as a brushroll light source and is directed toward the brushroll-receiving location 346, and the second light source 362 may be referred to as a recovery tank light source and is directed toward the recovery tank-receiving location 350.

With continued reference to FIG. 16, the brushroll 26 includes a first brushroll end 374 and a second brushroll end 378 opposite the first brushroll end 374. The first brushroll end 374 is proximal to and surrounded by the first light source 358, and the second brushroll end 378 is distal from the first light source 358 and is proximal to the first door 330. Similarly, the recovery tank 18 includes a first recovery tank end 382 and a second recovery tank end 386 opposite the first recovery tank end 382. The first recovery tank end 382 is proximal to and surrounded by the second light source 362, and the second recovery tank end 386 is distal from the second light source 362 and proximal to the second door 334. The light source 358, and accordingly the plurality of light sources 366, is directed away from the first brushroll end 374 toward the second brushroll end 378 such that ultraviolet light is directed along a length of the brushroll 26 onto and/or within the brushroll 26. Similarly, the light source 362, and accordingly the plurality of light sources 370, is directed away from the first recovery tank end 382 and toward the second recovery tank end 386 such that ultraviolet light is directed along a length of the recovery tank 18 to provide the desired cleaning or sanitizing effect for the application, such as, for one example, inhibiting microbial growth on and/or within the recovery tank 18. In the illustrated embodiment, the light sources 370 include one or more light sources 370 directing ultraviolet light toward the interior of a tank component, e.g., the recovery tank 18, and one or more light sources 370 directing ultraviolet light toward the exterior of the tank component. In some embodiments, the light source 362 directs light only to the interior of the tank component. In other embodiments, the light source 362 directs light only to the exterior of the tank component. The cleaning apparatus 28 may include a wiper configured along the axis A3, such as the wiper 216 illustrated in FIGS. 5-9, to expose an interior of the brushroll 26 to the ultraviolet light. Further, the light sources 358, 362, 366, 370 may collectively or individually be covered by a UV transmitting lens to shield the light sources 358, 362, 366, 370, for example, such as the lens 208 disclosed herein. Reflectors such as the reflectors 212 disclosed herein may reflect light from the light sources 358, 362, 366, 370 toward the removable components of the floor cleaner 10 that are to be cleaned. In the illustrated embodiment, the light sources 358, 362 are directed toward the upper side 328 of the housing 314 and away from the lower side 332 of the housing 314. In other embodiments, the light sources 358, 362 are arranged in other orientations as desired for the application, such as along the axes A3, A5, or directed toward the lower side 332 of the housing 314 and away from the upper side 328 of the housing 314, or other arrangements as desired to provide ultraviolet light onto the component as desired.

With continued reference to FIG. 16, the cleaning apparatus 310 includes a fan 390 as a cleaning member within the compartment 318. The fan 390 is powered by a motor 394 that transmits rotational torque through a driveshaft 398 such that a rotational axis A4 of the fan is parallel to and, more specifically, collinear with a rotational axis of the motor 394. The fan 390 is configured to direct air through an air outlet 402 along a desired airflow path 406 into the housing 314. The fan 390 may draw air through an air inlet (not shown) and exhaust through the air vents 338 (FIG. 15). The air vents 338 may be oriented to provide a flow of air along the tray 322 and the underside of the floor cleaner 10 when mounted in the tray 322 to facilitate drying of the underside of the floor cleaner 10 and/or tray 322. In the illustrated embodiment, the fan 390 is positioned between the brushroll-receiving location 346 and the recovery tank-receiving location 350, but in other embodiments, the fan 390 may be positioned in any position and orientation as desired to provide an amount of drying of the removable components of the floor cleaner 10 as desired for the application. In some embodiments, the airflow path 406 is directed or ducted toward the components configured to facilitate drying, for example, to the interior of the recovery tank 18. In some embodiments, an air distributor system is provided in the housing along the brushroll axis A3, such as any one or more of the features and embodiments described in this detailed description with respect to the air distributor system 246.

With continued reference to FIG. 16, a first sensor 410 is configured to determine when the first door 330 is positioned in the closed position and/or latched. A second sensor 414 is configured to determine when the second door 334 is positioned in the closed position and/or latched. The sensors 410, 414 provide inputs to control circuitry onboard the cleaning apparatus 310 or to the control circuitry 40 of the floor cleaner 10, and the control circuitry utilizes the inputs to, at least in part, determine whether to operate a cleaning cycle.

In operation, and with continued reference to FIGS. 15 and 16, removable components of the floor cleaner 10 such as, in the illustrated embodiment, the brushroll 26 and the recovery tank 18, are removed from the floor cleaner 10 and placed within the compartment 318 at the brushroll-receiving location 346 and the recovery tank-receiving location 350, respectively. The base 12 of the floor cleaner 10 is placed in the tray 322 in a mounted position for storage, for charging a battery onboard the floor cleaner 10, for operating a cleaning cycle to clean the base 12 in the tray 322, and/or for other purposes. The cleaning apparatus 310 is operable in a cleaning cycle of a cleaning mode to operate the light sources 358, 362 (and more specifically, the plurality of respective smaller light sources 366, 370) to emit ultraviolet light and to direct the ultraviolet light toward the brushroll 26 and toward the recovery tank 18. As disclosed herein, the cleaning apparatus 310 may include a wiper such as, for example, the wiper 216 (for example, see FIG. 9) that interferes with the brushroll 26 while the brushroll 26 is rotated during the cleaning cycle to expose an interior of the brushroll 26 to the ultraviolet light. Similarly, the wiper 216 may expose an interior of the brushroll 26 to the airflow path 406 to facilitate improved drying of the operational material 130 of the brushroll 26.

In operation, and with continued reference to FIGS. 15 and 16, a cleaning cycle of a cleaning mode may include operating the fan 390 to enhance cleaning and/or drying of components such as the brushroll 26 and/or the recovery tank 18. The fan 390 may operate simultaneously with the light sources 358, 362 or before or after the light sources 358, 362 operate. The sensors 410, 414 transmit a signal to control circuitry so that, in some embodiments, the cleaning cycle only operates when one or both of the doors 330, 334 are in the closed position.

A method of cleaning one or more components, such as the brushroll 26 or the recovery tank 18 of the floor cleaner 10, with the cleaning apparatus 310 includes removing the component from the floor cleaner 10 and placing the component in the compartment 318 of the cleaning apparatus 310. The method includes operating a light source such as the light sources 358, 362 to emit ultraviolet light and to direct the ultraviolet light toward the component. The method may include rotating the component to achieve relatively uniform application of ultraviolet light to the component. The rotational speed of the component may be selected to achieve a desired dwell time of the ultraviolet light on the component. For example, the method includes operating a light source to direct ultraviolet light toward an exposed interior of a brushroll 26 of the floor cleaner 10 and rotating the brushroll 26 at a desired rotational speed to achieve a desired dwell time of the ultraviolet light in the brushroll 26. The method may further include directing the ultraviolet light toward the component with a reflector such as the reflector 212, which in some embodiments forms a concave shape or angled shape or other shape adjacent the light source configured by its shape and surface properties to reflect ultraviolet light. The method may further comprise sensing that the floor cleaner 10 is in a mounted position, such as in a mounted position in the tray 322, and the sensing may be performed by control circuitry in the cleaning apparatus 310 or by the control circuitry 40 in the floor cleaner 10. The method may further include activating a cleaning mode when the control circuitry (e.g., control circuitry in the cleaning apparatus 310 or the control circuitry 40 in the floor cleaner 10) senses that the floor cleaner 10 is in the mounted position in the tray 322. The method may further include wiping the brushroll 26 with a wiper such as the wiper 216 while the brushroll 26 is rotating inside the compartment 318 to expose an interior of the operational material 130 of the brushroll 26 to the ultraviolet light emitted by one or more of the light sources. The method may include engaging a wiper 216 against the operational material 130 of the brushroll 26 adjacent the light source to provide ultraviolet light inside the outer perimeter 126 of the brushroll 26. The method may further include shielding the light source from moisture and debris with a lens. In one embodiment, the wiper may be an extension of the lens. The method may further include sensing whether a door (e.g., one or more of the doors 330, 334) is positioned in the closed position with a sensor, the sensor configured to generate a position signal corresponding to whether the door 330, 334 is positioned in the closed position. The method may further include transmitting the position signal to control circuitry (e.g., control circuitry in the cleaning apparatus 310 or the control circuitry 40 in the floor cleaner 10) and controlling an operating of a light source with the control circuitry based at least in part on the position signal, wherein the control circuitry is configured to operate the light source for a desired length of time. The method may further include latching one or more of the doors 330, 334 in the closed position to selectively retain the doors 330, 334 in the closed position. The method may further include sensing whether the doors 330, 334 are latched in the closed position and transmitting a latch signal to control circuitry (e.g., control circuitry in the cleaning apparatus or the control circuitry 40 in the floor cleaner 10), the latch signal corresponding to whether the at least one of the doors 330, 334 is latched in the closed position, and determining whether to operate a cleaning cycle based at least in part on the latch signal.

Embodiments disclosed herein may be combined in a series of permutations to form yet other embodiments.

Various features and advantages of the disclosure are set forth in the following claims.

1. A combination of a floor cleaner and a cleaning apparatus for cleaning the floor cleaner, the combination comprising:
a floor cleaner including:
a base movable along a surface to be cleaned,
a brushroll motor, and
a brushroll having a length along a brushroll axis, the brushroll rotatable about the brushroll axis relative to the base by the brushroll motor, the brushroll having a brushroll radius defined from the brushroll axis to an outer perimeter of the brushroll,
a cleaning apparatus including a tray configured to selectively removably receive a portion of the floor cleaner in a mounted position, the tray including a brushroll-receiving portion to receive the brushroll of the floor cleaner, and
a light source directed toward the brushroll when the brushroll is received in the brushroll-receiving portion of the tray, the light source operable to emit ultraviolet light having a wavelength less than 400 nm.

2. The combination of claim 1, further comprising a wiper configured to engage the outer perimeter of the brushroll adjacent the light source.

3. The combination of claim 2, wherein the wiper is positioned a radial first distance from the brushroll axis when the floor cleaner is in the mounted position, and wherein the radial first distance is less than the brushroll radius such that the wiper interferes with the brushroll when the floor cleaner is in the mounted position.

4. The combination of claim 3, wherein the radial first distance is between 0.5 mm and 12 mm less than the brushroll radius.

5. The combination of claim 3, wherein the brushroll includes an operational material having fibers having a fiber length, and the difference between the radial first distance and the brushroll radius is a function of the fiber length.

6. The combination of claim 2, wherein the wiper is configured to move a portion of the brushroll to expose an interior of the brushroll to the ultraviolet light.

7. (canceled)

8. The combination of claim 2, further comprising a transparent lens positioned between the light source and the brushroll when the floor cleaner is in the mounted position.

9. The combination of claim 8, wherein the wiper is an extension of the lens.

10.-13. (canceled)

14. The combination of claim 2, wherein the wiper extends longitudinally along at least a portion of a length of the brushroll-receiving portion of the tray, the wiper configured to interfere with the length of the brushroll.

15.-23. (canceled)

24. The combination of claim 1, wherein the light source includes a light-emitting diode.

25. The combination of claim 1, wherein the light source extends longitudinally along at least a portion of a length of the brushroll-receiving portion of the tray, the light source configured to emit the ultraviolet light onto the length of the brushroll.

26. The combination of claim 1, further comprising control circuitry housed inside the tray and electrically connected to the light source to control an operation of the light source.

27. The combination of claim 1, wherein the tray includes an actuator configured to activate the brushroll when the floor cleaner is in the mounted position.

28. The combination of claim 1, wherein the floor cleaner includes control circuitry that controls the brushroll, the control circuitry operable in a first condition including a brushroll-enabled condition when the floor cleaner is in an operational condition, a second condition including a brushroll-disabled condition when the floor cleaner is in a storage condition and the floor cleaner is not in the mounted position, and a third condition including a brushroll-enabled condition when the floor cleaner is in the mounted position.

29. The combination of claim 28, wherein the cleaning apparatus includes an actuator that activates the control circuitry into the third condition, and wherein the control circuitry enables a cleaning mode in the third condition.

30. The combination of claim 29, wherein the cleaning mode includes operation of the light source.

31. The combination of claim 1, wherein a cleaning mode is operable in response to a user activation when the floor cleaner is in the mounted position.

32. The combination of claim 26, wherein the control circuitry is operable in response to a user activation.

33.-34. (canceled)

35. The combination of claim 1, further comprising a fan selectively operable to direct air through an air distributor having a plenum and one or more air openings directed toward the brushroll.

36.-38. (canceled)

39. The combination of claim 35, further comprising control circuitry to operate the fan when the floor cleaner is in the mounted position.

40.-71. (canceled)
